(19) United States
(12) Patent Application Publication
(10) Pub. No.: US 2021/0098123 A1
(43) Pub. Date: Apr. 1, 2021

(54) MEDICAL SUPPORT APPARATUS, INFORMATION PROCESSOR, AND MEDICAL SUPPORT METHOD

(71) Applicant: OLYMPUS CORPORATION, Tokyo (JP)

(72) Inventor: Yoshihide ENDO, Tokyo (JP)

(73) Assignee: OLYMPUS CORPORATION, Tokyo (JP)

(21) Appl. No.: 17/118,926

(22) Filed: Dec. 11, 2020

Related U.S. Application Data
(63) Continuation of application No. PCT/JP2019/007057, filed on Feb. 25, 2019.

(30) Foreign Application Priority Data
Jun. 12, 2018 (JP) 2018-112117

(51) Int. Cl. G16H 40/40; A61B 1/00

(52) U.S. Cl. CPC G16H 40/40; A61B 1/00059; A61B 1/00055

(57) ABSTRACT

A failure information memory unit stores information regarding a period until the failure of an endoscope in a plurality of medical facilities for each cause of failure. A similar facility identification unit identifies a second medical facility similar to a first medical facility. A date determination unit determines, using information regarding a period until failure of an endoscope in the second medical facility, designated date information regarding a date on which alerting should be made in the first medical facility according to the cause of the failure. A transmitter transmits the combination of the determined designated date information and information regarding the cause of the failure to the first medical facility.

### CROSS-REFERENCE TO RELATED APPLICATIONS

This application is based upon and claims the benefit of priority from the prior Japanese Patent Applications No.2018-112117, filed on Jun. 12, 2018, and International Application No. PCT/JP2019/007057, filed on Feb. 25, 2019, the entire contents of which are incorporated herein by reference.

### BACKGROUND OF THE INVENTION

### 1. Field of the Invention

The present disclosure relates to medical support apparatuses that collect information on failed endoscopes in a plurality of medical facilities and to information processors provided in the medical facilities.

### 2. Description of the Related Art

Patent Document 1 discloses an attention calling device having: a detecting means that detects abnormal states caused due to a user operation; and an attention calling means for alerting the user in order to avoid the occurrence of abnormal states at the time of a new user operation.

[Patent Document 1] Japanese Patent Application Publication No. 2004-128731

As a result of analyzing the causes of endoscope failure, it has been found that 40 percent or more of the failure is due to human error. One of the measures for reducing human error is to present the worker with cautionary information regarding the causes of endoscope failure before work. However, if the frequency of alerting is too high, the worker may become accustomed to the presentation of cautionary information, and as a result, may not see the cautionary information.

### SUMMARY OF THE INVENTION

In this background, a purpose of the present disclosure is to provide a technology for efficiently presenting cautionary information regarding the causes of endoscope failure to the worker.

An embodiment of the present disclosure relates to a medical support apparatus including: a processor including hardware; and a memory that stores information regarding a period until failure of an endoscope in a plurality of medical facilities for each cause of failure, wherein the processor is configured to: identify a second medical facility similar to a first medical facility; use information regarding a period until failure of an endoscope in the second medical facility so as to determine designated date information regarding a date on which alerting should be made in the first medical facility according to the cause of the failure; and transmit the combination of the determined designated date information and information regarding the cause of the failure to the first medical facility.

Another embodiment of the present disclosure relates to an information processor provided in a first medical facility, including: a processor including hardware, wherein the processor is configured to: acquire the combination of designated date information regarding a date on which alerting should be made in the first medical facility and information regarding the cause of the failure, the designated date information which is determined by a medical support apparatus by using information regarding a period until failure of an endoscope in a second medical facility similar to the first medical facility; and present cautionary information regarding the cause of the failure to a worker on a date specified by the acquired designated date information.

Yet another embodiment of the present disclosure relates to a medical support method including: storing information regarding a period until failure of an endoscope in a plurality of medical facilities for each cause of failure; identifying a second medical facility similar to a first medical facility; using information regarding a period until failure of an endoscope in the second medical facility so as to determine designated date information regarding a date on which alerting should be made in the first medical facility according to the cause of the failure; and transmitting the combination of the determined designated date information and information regarding the cause of the failure to the first medical facility.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the entire configuration of an information processing system in an embodiment;

FIG. 2 is a diagram showing examples of the causes of endoscope failure due to human error;

FIG. 3 is a diagram showing functional blocks of a medical support apparatus;

FIG. 4 is a diagram showing an example of failure information stored in a failure information memory unit;

FIG. 5 is a diagram showing an example of status information stored in a facility status memory unit;

FIG. 6 is a diagram showing functional blocks of an information processor;

FIG. 7 is a diagram showing examples of failure information of predetermined medical facilities; FIG. 8 is a diagram showing alerting dates that are managed;

FIG. 9 is a diagram showing examples of timing for presenting precautions for the causes of endoscope failure;

FIG. 10 is a diagram showing an example of cautionary information presented to a nurse;

FIG. 11 is a diagram showing an example of cautionary information presented to a doctor; and

FIG. 12 is a diagram showing an example of cautionary information presented to a nurse.

### DETAILED DESCRIPTION OF THE INVENTION

The invention will now be described by reference to the preferred embodiments. This does not intend to limit the scope of the present invention, but to exemplify the invention.

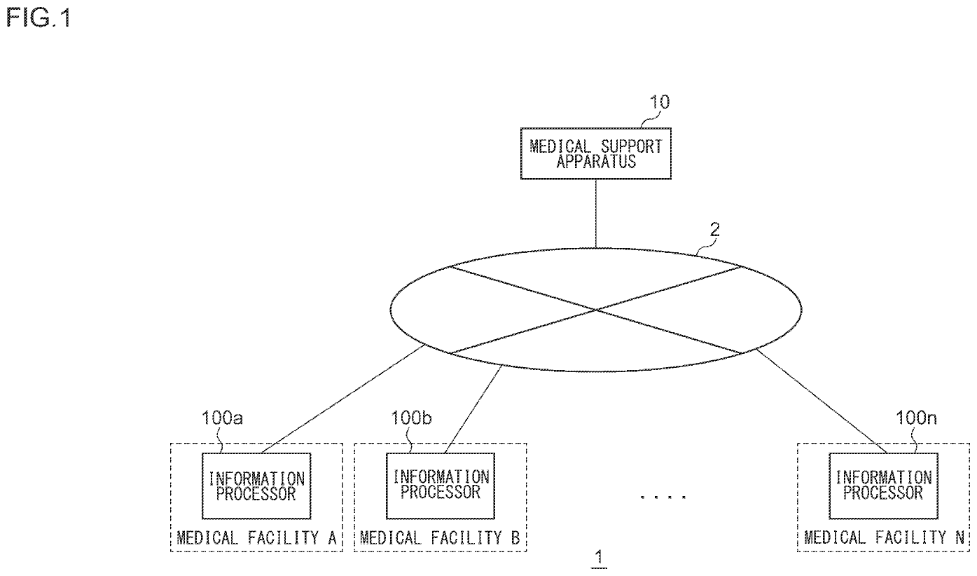

FIG. 1 shows the entire configuration of an information processing system 1 in an embodiment of the present disclosure. The information processing system 1 includes a medical support apparatus 10 and information processors 100a, 100b, . . . , and 100n (hereinafter, when not particularly distinguished, referred to as “information processors 100”) provided at a plurality of medical facilities. In a case of failure of endoscope, each medical facility sends the failed endoscope to the endoscope manufacturer for repair. The medical support apparatus 10 according to the embodiment is operated and managed by an endoscope manufacturer, and collects information on a failed endoscope.

The information processors 100 according to the embodiment have a function of presenting cautionary information regarding the causes of endoscope failure to the worker, and the medical support apparatus 10 determines the date of presentation of the cautionary information by each information processor 100 for each cause of failure. Therefore, in the information processing system 1, the medical support apparatus 10 operates as a server device, and the information processors 100 operate as client devices. The information processor 100 transmits a request for acquiring the date of presentation to the medical support apparatus 10, and acquires the date of presentation from the medical support apparatus 10. The date of presentation of the cautionary information is appropriately determined for each medical facility, and the information processor 100 presents the cautionary information to the worker involved in the cause of failure at the timing determined by the medical support apparatus 10. The cautionary information includes information that specifically indicates the action that becomes the cause of failure, and the worker can check the matters to be attended to by looking at the cautionary information.

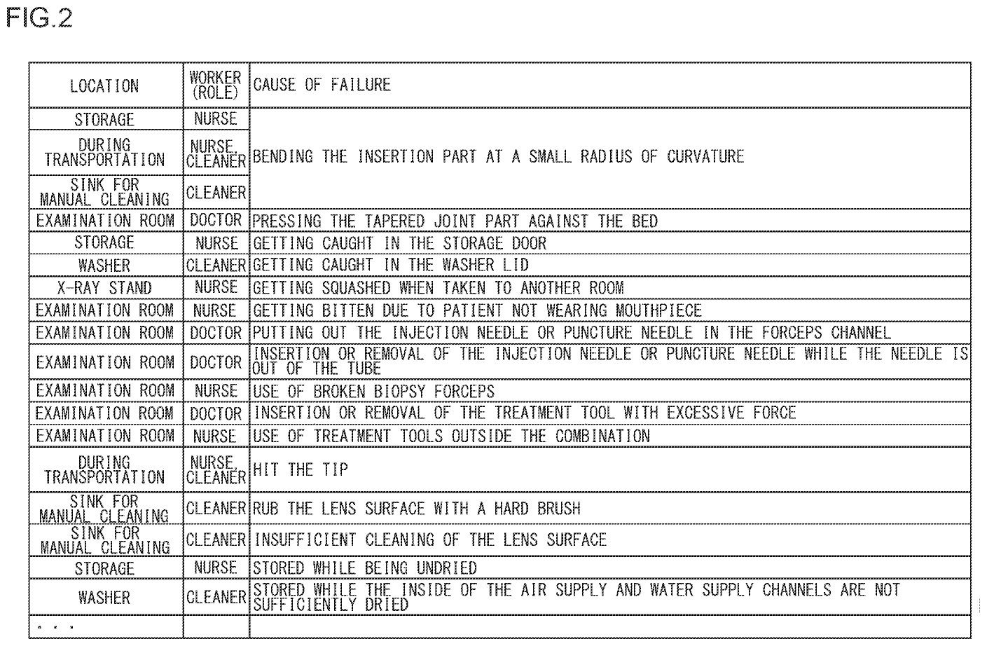

FIG. 2 shows examples of the causes of endoscope failure due to human error. Endoscope failure caused by “bending the insertion part at a small radius of curvature” occurs due to carelessness of the nurse or the cleaner. The failure occurs in a storage, during transportation, and in a sink for manual cleaning. Endoscope failure caused by “pressing the tapered joint part against the bed” occurs in an examination room due to carelessness of the doctor. Endoscope failure caused by “getting caught in the storage door” occurs in the storage due to carelessness of the nurse. Endoscope failure caused by “getting caught in the washer lid” occurs in the washer due to carelessness of the cleaner.

Since there are various causes for endoscope failures, in order to reduce human error, specific cautionary information regarding the causes of failure need to be presented to the appropriate worker at an appropriate place and at an appropriate time. For example, in order to reduce the frequency of endoscope failure caused by “getting caught in the storage door”, the information processor 100 preferably presents cautionary information indicating “Please be careful not to get the endoscope caught in the storage door when storing the endoscope” to the nurse before the first storage work of the day in the storage. It is meaningless to present this cautionary information to doctors or cleaners who are not in charge of storage work, and it is not effective to present the cautionary information outside the installation location in the storage.

On the other hand, if the cautionary information is presented each time the work is performed, the worker may feel annoyed and may not see the cautionary information. Therefore, it is considered effective to present cautionary information to the worker at an appropriate frequency. The present discloser has focused on the fact that the period until failure due to each cause is different, and devised a technique for deriving a presentation frequency suitable for each medical facility for each cause of failure according to the situation of each medical facility.

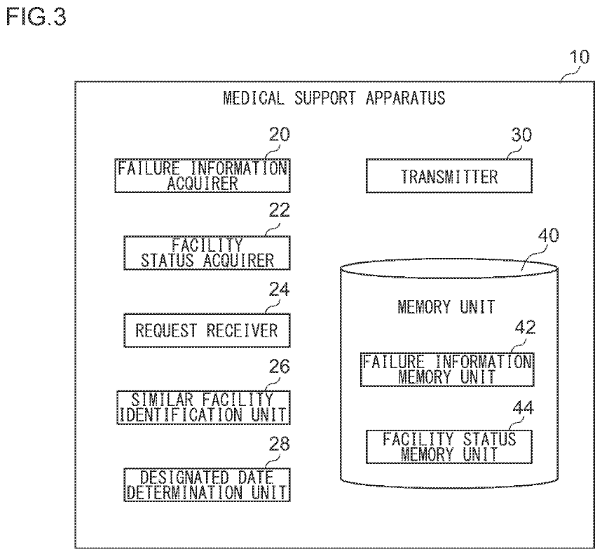

FIG. 3 shows functional blocks of a medical support apparatus 10. The medical support apparatus 10 includes a failure information acquirer 20, a facility status acquirer 22, a request receiver 24, a similar facility identification unit 26, a designated date determination unit 28, a transmitter 30, and a memory unit 40. The memory unit 40 has a failure information memory unit 42 and a facility status memory unit 44. Each feature of a medical support apparatus 10 is implemented by hardware such as a processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The failure information acquirer 20 acquires failure information of endoscopes that have failed in each medical facility. As the failure information, the failure information acquirer 20 acquires at least model information for identifying the type of a failed endoscope, worker type information for identifying the type of a worker who has caused the endoscope to fail, and the cause of the failure. In the embodiment, the worker type information is identification information for distinguishing a doctor, a nurse, and a cleaner. Further, in a case where a cause ID for uniquely identifying each of causes of failure shown in FIG. 2 is assigned, the failure information may be the cause ID of the corresponding cause of failure.

When an endoscope fails, the information processor 100 may transmit identification information (scope ID) for the failed endoscope, model information for the endoscope, worker type information, and the cause of failure (cause ID) to the medical support apparatus 10 via a network 2 along with identification information (facility ID) for identifying the medical facility. The information processor 100 has a predetermined medical support application that has been installed, and the person in charge of the medical facility starts the medical support application, displays a transmission format on a display of the information processor 100, and enters failure information in the transmission format. When the information processor 100 transmits the facility ID and the failure information to the medical support apparatus 10 via the network 2, the failure information acquirer 20 acquires the facility ID and the failure information transmitted from the information processor 100 and stores the facility ID and the failure information in the failure information memory unit 42.

When the information processor 100 does not transmit the failure information in a predetermined format, the entry of the facility ID and the failure information into the medical support apparatus 10 by the repairman of the endoscope manufacturer may cause the failure information acquirer 20 to acquire the facility ID and the failure information and store the facility ID and the failure information in the failure information memory unit 42.

When an endoscope fails, the person in charge of the medical facility informs the endoscope manufacturer of the failure by telephone, email, or the like, and sends the failed endoscope to the endoscope manufacturer. If the person in charge notifies the endoscope manufacturer of the situation, cause, and the like of the failure at this time, the repairman of the endoscope manufacturer enters the failure cause and the like as notified by the person in charge of the medical facility into the medical support apparatus 10, thereby allowing the failure information acquirer 20 to acquire the facility ID and the failure information. Even if the person in charge of the medical facility cannot tell the cause of the failure, the repairman may guess the cause of the failure and the like based on the situation of the failure of the endoscope that has been sent and enter the guesses cause of the failure into the medical support apparatus 10. In any case, the failure information acquirer 20 acquires failure information including the cause of failure of the endoscope that has failed in each medical facility and stores the failure information in the failure information memory unit 42.

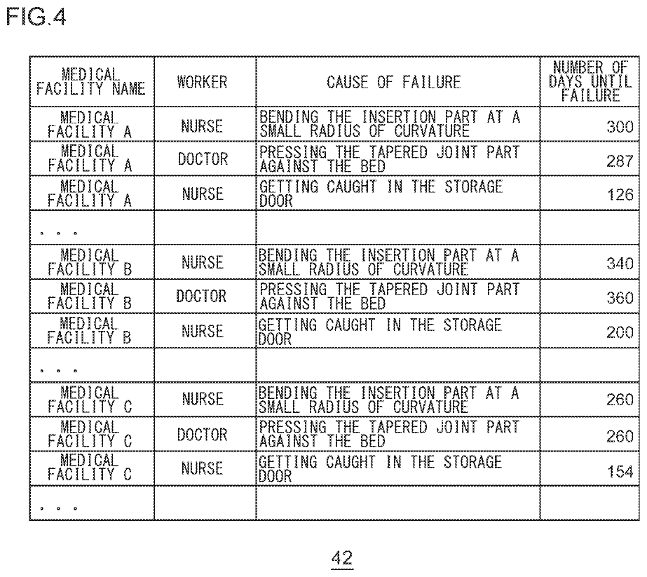

FIG. 4 shows an example of failure information stored in the failure information memory unit 42. For convenience, FIG. 4 shows only the failure information of medical facilities A to C. However, the actual failure information memory unit 42 also stores the failure information of other medical facilities included in the information processing system 1. FIG. 4 shows the names of the medical facilities, workers included in the failure information, the causes of failure, and the model information, etc., of failed endoscopes are omitted.

The failure information memory unit 42 stores information regarding a period until the failure of an endoscope in a plurality of medical facilities for each cause of failure. In this example, “the number of days until failure” is stored as information regarding the period until failure. In this case, “the number of days until failure” represents the number of days from a predetermined starting date to the closest (latest) failure date to the current date. The starting date may be the date when a failure due to the same cause occurred last time before the latest failure date, and in this case, “the number of days until failure” is the interval between the two recent failure dates due to the same cause. Further, the starting date may be the date when cautionary information regarding the cause is presented at the medical facility, and in this case, “the number of days until failure” represents the interval from the latest alerting date to the latest failure date. The starting date of “the number of days until failure” may be either the last failure date before the latest failure date or the latest alerting date. However, in the following, the starting date is the latest alerting date.

For example, in the medical facility A, 300 days have passed from the alerting date on which the nurse was presented with cautionary information regarding “bending the insertion part at a small radius of curvature” to the date on which the nurse caused a failure due to “bending the insertion part at a small radius of curvature”. This means that at the medical facility A, by presenting the cautionary information regarding “bending the insertion part at a small radius of curvature” to the nurse, the nurse was careful not to bend the insertion part at a small radius of curvature, and no failure due to the cause occurred for 299 days. That is, in the medical facility A, the effect of presenting the cautionary information regarding “bending the insertion part at a small radius of curvature” lasted for 299 days.

In the same way, in the medical facility A, 126 days have passed from the alerting date on which the nurse was presented with cautionary information regarding “getting caught in the storage door” to the date on which the nurse caused a failure due to “getting caught in the storage door”. This means that at the medical facility A, by presenting the cautionary information regarding “getting caught in the storage door” to the nurse, the nurse was careful not to cause endoscopes to get caught in the storage door, and no failure due to the cause occurred for 125 days. That is, in the medical facility A, the effect of presenting the cautionary information regarding “getting caught in the storage door” lasted for 125 days.

In this way, “the number of days until failure—1” can be considered as the number of days for which the effect of presenting the cautionary information lasted. The failure information memory unit 42 stores the number of days until failure in a plurality of medical facilities for each cause of failure. When the failure information acquirer 20 acquires failure information, the failure information memory unit 42 updates the corresponding failure information. Therefore, the alerting date for each cause of failure in each medical facility is stored in the memory unit 40, and the failure information acquirer 20 calculates the number of days from the latest alerting date to the failure and stores the calculated number of days in the failure information memory unit 42.

The facility status acquirer 22 acquires status information of each medical facility. The status information is information that shows the scale and characteristics of a medical facility regarding endoscopic examinations. The characteristics of the medical facility include the types of examinations that can be performed, and the like. The facility status acquirer 22 acquires the number of endoscopes that can be used in each medical facility and model information indicating the types of endoscopes that can be used, and stores the number of endoscopes and the model information in the facility status memory unit 44. The number of endoscopes that can be used is information that represents the scale of the medical facility, and the model information of the endoscopes that can be used is information that represents the characteristics of the medical facility, that is, the types of examinations that can be performed.

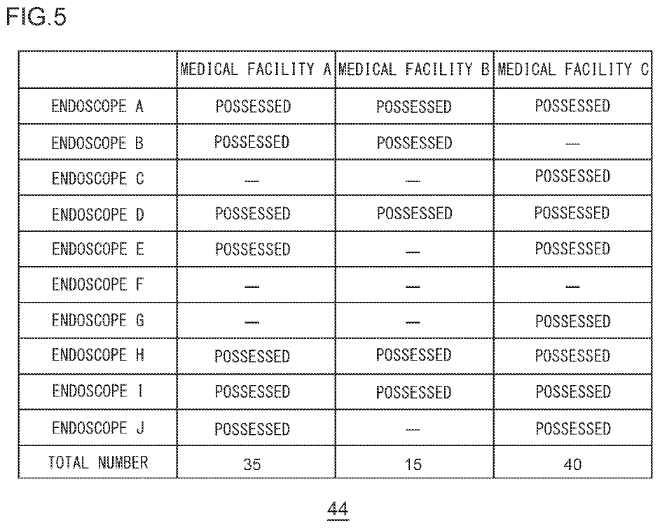

FIG. 5 shows an example of status information stored in the facility status memory unit 44. For convenience, FIG. 5 shows only the status information of the medical facilities A to C. However, the actual facility status memory unit 44 also stores the status information of other medical facilities included in the information processing system 1. In the embodiment, the facility status memory unit 44 stores the number of endoscopes that can be used in a medical facility and the model information of the endoscopes that can be used.

The facility status memory unit 44 according to the embodiment stores the possession status of ten types of endoscope models A to J and the total number of endoscopes that can be used in the medical facilities. In this example, the medical facility A has the endoscope models A, B, D, E, H, I, and J, and does not possess the endoscope models C, F, or G. The medical facility B has endoscope models A, B, D, H, and I, and does not possess endoscope models C, E, F, G, or J. The medical facility C has endoscope models A, C, D, E, G, H, I, and J, and does not possess endoscope models B or F.

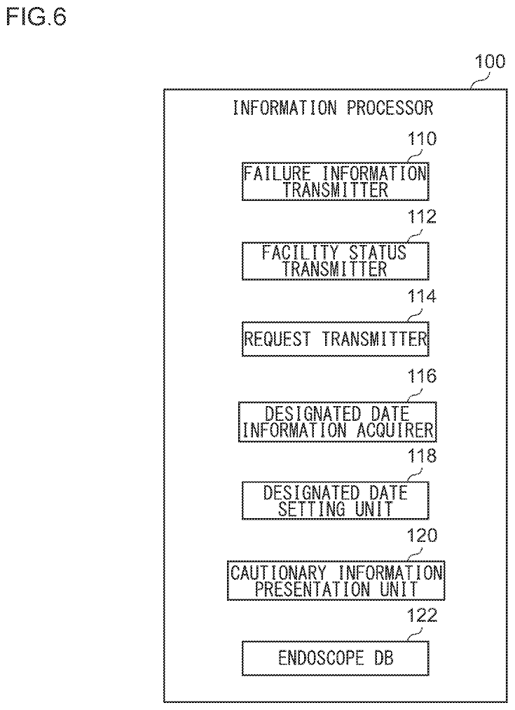

FIG. 6 shows functional blocks of an information processor 100 provided in each medical facility. The information processor 100 includes a failure information transmitter 110, a facility status transmitter 112, a request transmitter 114, a designated date information acquirer 116, a designated date setting unit 118, a cautionary information presentation unit 120, and an endoscope DB 122. The endoscope DB 122 is a database for registering information on endoscopes that can be used in the medical facility, and stores identification information and model information of the endoscopes that the medical facility has.

Each feature of the information processor 100 is implemented by hardware such as a processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

When an endoscope fails, the person in charge of the medical facility starts the medical support application, displays a transmission format on a display of the information processor 100, and enters failure information in the transmission format. The failure information to be entered includes the scope ID and model information of the failed endoscope, the type information of a worker who caused the endoscope to fail, and the cause of the failure. The failure information transmitter 110 transmits the entered failure information to the medical support apparatus 10 via the network 2 along with the facility ID.

The facility status transmitter 112 refers to the stored content of the endoscope DB 122 as the status information of the medical facility, acquires the number of endoscopes that can be used in the medical facility and the model information of the endoscopes that can be used, and transmits the number of endoscopes and the model information to the medical support apparatus 10 via the network 2 along with the facility ID. Although the facility status transmitter 112 may periodically transmit status information, the facility status transmitter 112 may transmit status information when the endoscope DB 122 is updated.

The request transmitter 114 transmits a request for acquiring the date of presentation of cautionary information to the medical support apparatus 10. In the information processing system 1, the date of presentation of cautionary information for each cause of failure is determined by the medical support apparatus 10, and the information processor 100 of each medical facility presents the cautionary information to the persons involved on the determined date. Therefore, on the day when cautionary information regarding the cause of failure is presented at the medical facility, the request transmitter 114 transmits a request for acquiring the next presentation date of the cautionary information regarding the cause of failure to the medical support apparatus 10. The request transmitter 114 transmits a request for acquiring the next presentation date to the medical support apparatus 10 on the day when the cautionary information is presented at the medical facility, thereby allowing the designated date information acquirer 116 to acquire designated date information that is ideal at that time.

In the medical support apparatus 10, the request receiver 24 receives a request for acquiring designated date information regarding a date on which alerting should be made from the information processor 100. The designated date determination unit 28 determines designated date information regarding an alerting date. At this time, the designated date determination unit 28 uses failure information in another medical facility similar to the medical facility that has transmitted the acquiring request so as to determine the designated date information.

In the information processing system 1, there are medical facilities of various scales and characteristics. The designated date determination unit 28 determines designated date information regarding a date on which alerting should be made for each cause of failure. At this time, the designated date determination unit 28 can determine more appropriate designated date information by referring to failure information in a plurality of medical facilities. Therefore, in the embodiment, the similar facility identification unit 26 identifies another medical facility similar to the medical facility that has transmitted the acquiring request, and the designated date determination unit 28 determines designated date information in the medical facility that has transmitted the acquiring request by using information regarding a period until the failure of an endoscope in the other similar medical facility.

The similar facility identification unit 26 identifies the medical facility based on the facility ID included in the acquiring request, and searches for another medical facility similar to the medical facility. More specifically, the similar facility identification unit 26 calculates the similarity between the facility and other medical facilities with reference to the facility status stored in the facility status memory unit 44, and identifies a medical facility with a high degree of calculated similarity.

Hereinafter, an example of a technique for calculating the degree of similarity between the medical facility A and other medical facilities will be described using the status information shown in FIG. 5. In the embodiment, the degree of similarity between medical facilities is calculated based on the matching degree regarding the scale of the medical facilities and the matching degree regarding the characteristics of the medical facilities. The matching degree regarding the scale of the medical facilities is calculated as the matching degree regarding the number of endoscopes that can be used at the medical facilities, and the matching degree regarding the characteristics of the medical facilities is calculated as the matching degree regarding the types of endoscopes that can be used.

<Matching Degree Regarding the Number of Endoscopes>

The similar facility identification unit 26 calculates the matching degree regarding the number of endoscopes by the following Expression 1.

$$1 - \vert \frac{( \begin{matrix}NUMBER OF \\ ENDOSCOPES IN \\ FACILITY A\end{matrix} ) - ( \begin{matrix}NUMBER OF \\ ENDOSCOPES \\ IN ANOTHER \\ FACILITY\end{matrix} )}{( \begin{matrix}NUMBER OF ENDOSCOPES IN \\ FACILITY A OR NUMBER OF \\ ENDOSCOPES IN ANOTHER \\ FACILITY , WHICHEVER \\ IS LARGER\end{matrix} )} \vert \qquad [ Expression 1 ]$$

According to Expression 1, the respective matching degrees regarding the number of endoscopes with respect to the medical facility B and with respect to the medical facility C are calculated as follows.

The matching degree regarding the number of endoscopes in the medical facility B: 0.43

The matching degree regarding the number of endoscopes in the medical facility C: 0.86

<Matching Degree Regarding the Types of Endoscopes>

The similar facility identification unit 26 derives the matching degree regarding the models possessed and the models not possessed between the medical facility A and the other medical facilities. Referring to the models in the medical facilities A and B in FIG. 5, although the medical facility A possesses a model E endoscope, the medical facility B does not possess a model E endoscope. Further, although the medical facility A possesses a model J endoscope, the medical facility B does not possess a model J endoscope. For other models, the state of possession and non-possession is the same between medical facilities A and B. Therefore, the medical facilities A and B match on the possession or non-possession of eight types of models out of ten types and do not match on the possession or non-possession of two types of models. Therefore, the similar facility identification unit 26 derives the matching degree regarding the types of endoscopes that can be used in medical facilities A and B to be 0.8.

Further, the medical facilities A and C match on the possession or non-possession of seven types (A, D, E, F, H, I, J) of models and do not match on the possession or non-possession of three types (B, C, G) of models. Therefore, the similar facility identification unit 26 derives the matching degree regarding the types of endoscopes that can be used in medical facilities A and C to be 0.7.

The matching degree regarding the types of endoscopes in the medical facility B: 0.8

The matching degree regarding the types of endoscopes in the medical facility C: 0.7

Once the similar facility identification unit 26 derives the matching degree regarding the number and the matching degree regarding the types between the medical facility A and another medical facility, the similar facility identification unit 26 multiplies the matching degrees of the two so as to calculate the degree of similarity between the facilities. The respective degrees of inter-facility similarity to the medical facility B and to the medical facility C are calculated as follows.

The degree of inter-facility similarity to the medical facility B: 0.34

The degree of inter-facility similarity to the medical facility C: 0.60

A high degree of inter-facility similarity indicates that the scale and characteristics of the facilities are similar, and therefore it is presumed that the frequency of the occurrence of failure for each cause of failure is also similar. In this example, the medical facility C is determined to be more similar to the medical facility A than the medical facility B.

In the above example, the similar facility identification unit 26 simply multiplies the matching degree regarding the number and the matching degree regarding the types so as to calculate the degree of inter-facility similarity. Alternatively, the similar facility identification unit 26 may change the weight of each matching degree so as to obtain the degree of inter-facility similarity. Since it is a common practice for medical facilities to construct an endoscopic examination environment according to the scale thereof, the weight of the matching degree regarding the number of endoscopes, which represents the scale of the facility, may be increased to be higher than the weight of the matching degree regarding the types of the endoscopes.

As described, the similar facility identification unit 26 uses the number of endoscopes that can be used in the medical facilities and the model information of the endoscopes that can be used so as to calculate the degree of similarity between a medical facility that has transmitted a request for acquiring designated date information regarding an alerting date and other medical facilities and identify a plurality of medical facilities with a high calculated degree of similarity. For example, the similar facility identification unit 26 may identify a predetermined number of medical facilities in descending order of the degree of inter-facility similarity, or may identify a plurality of medical facilities whose degree of inter-facility similarity exceeds a predetermined threshold value.

The designated date determination unit 28 determines the designated date information regarding an alerting date according to the cause of failure, using information regarding a period until the failure of an endoscope in a similar facility. The following is an explanation regarding a case where medical facilities C, E, and G are identified as similar facilities to the medical facility A and a request for acquiring an alerting date for a failure caused by “getting caught in the storage door” is transmitted from the medical facility A.

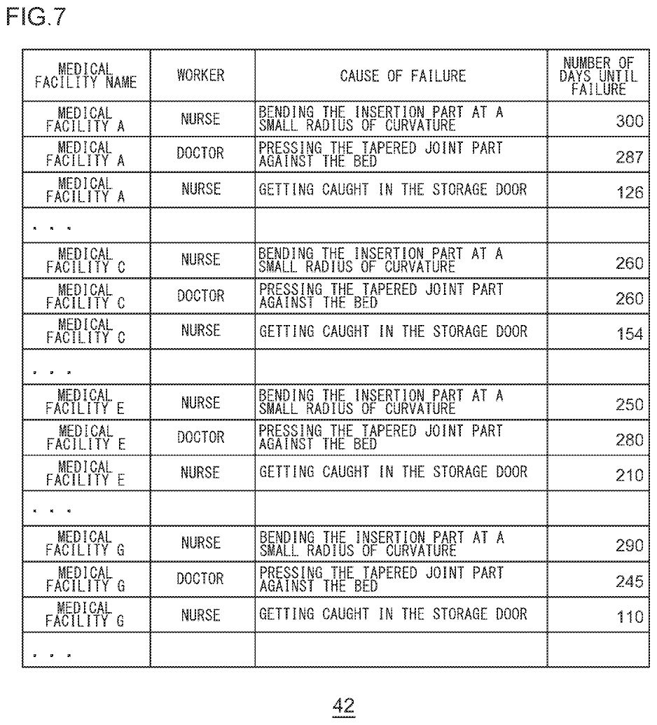

FIG. 7 shows an example of failure information of a predetermined medical facility stored in the failure information memory unit 42. FIG. 7 shows the failure information of the medical facility A that has transmitted a request for acquiring an alerting date and the failure information of the medical facilities C, E, and G similar to the medical facility A. The designated date determination unit 28 refers to the failure information of the medical facilities A, C, E, and G, and determines the designated date information regarding the alerting date for a failure caused by “getting caught in the storage door”.

With reference to FIG. 7, the number of days until a failure caused by “getting caught in the storage door” is stored as follows.

Medical facility A: 126 days

Medical facility C: 154 days

Medical facility E: 210 days

Medical facility G: 110 days

In the medical facility A, the number of days from the date on which the previous cautionary information was presented to the date on which the failure occurred is 126 days, and the effect of presenting the previous cautionary information therefore lasted for 125 days. The shortest number of days until failure is 110 days at the medical facility G, and the period in which the effect of presenting cautionary information lasted is 109 days.

Out of the number of days until failure stored with respect to the medical facility A and the similar medical facilities C, E, and G, the designated date determination unit 28 according to the embodiment uses the shortest number of days so as to determine designated date information regarding the next alerting date in the medical facility A. The designated date information may be determined as a period until the next alerting date. The designated date determination unit 28 determines (the shortest number of days in a similar facility—1) as the designated date information, and therefore determines the date on which alerting should be made regarding “getting caught in the storage door” to be 109 days later in this example. The transmitter 30 transmits the combination of the determined designated date information and information regarding the cause of failure to the information processor 100 of the medical facility A that has transmitted the request for acquiring the alerting date.

In the information processor 100, the designated date information acquirer 116 acquires the combination of the designated date information regarding the date on which alerting should be made and the information regarding the cause of failure from the information processor 100. The designated date setting unit 118 manages the previous alerting date and the number of days until the next alerting date.

FIG. 8 shows alerting dates managed by the designated date setting unit 118. The designated date setting unit 118 manages the previous alerting date and the number of days until the next alerting date from the previous alerting date. The “number of days until the next alerting date” is the number of days indicated by the designated date information transmitted from the medical support apparatus 10.

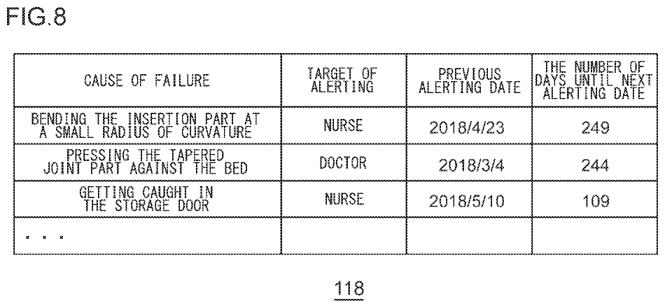

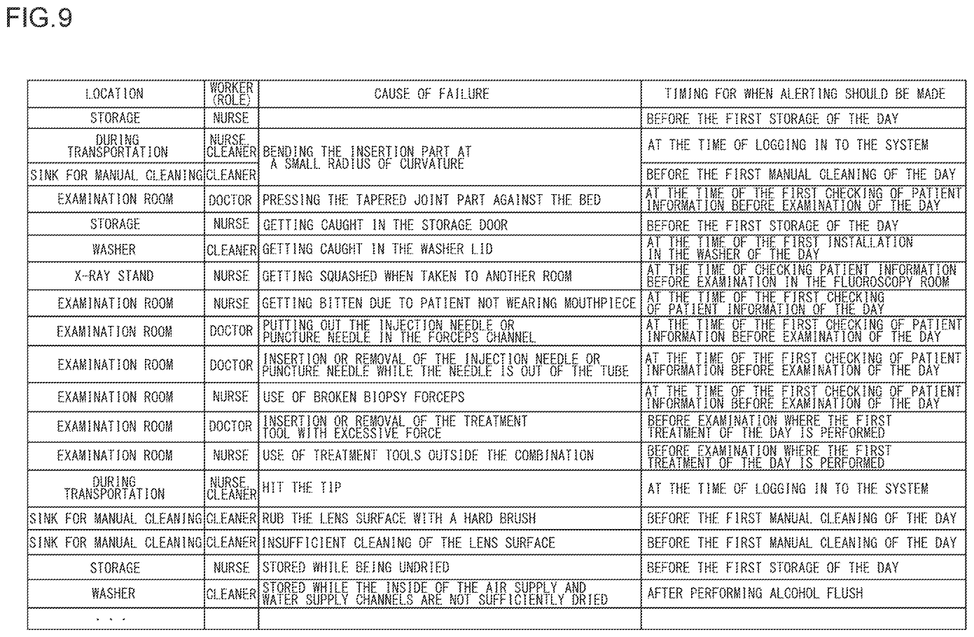

FIG. 9 shows examples of timing for presenting precautions for the causes of endoscope failure. This presentation timing is set to the time when the first work of the day is performed.

The cautionary information presentation unit 120 presents cautionary information regarding the cause of failure to workers who are involved on the date specified by the designated date information. On Jan. 24, 2019, which is 249 days after Apr. 23, 2018, the cautionary information presentation unit 120 presents cautionary information regarding “bending the insertion part at a small radius of curvature” to all nurses and cleaners at the medical facility A. The timing of presentation may be when the nurses and the cleaners first log in to the system, when the nurses first store an endoscope in the storage, or when the cleaners first perform manual cleaning.

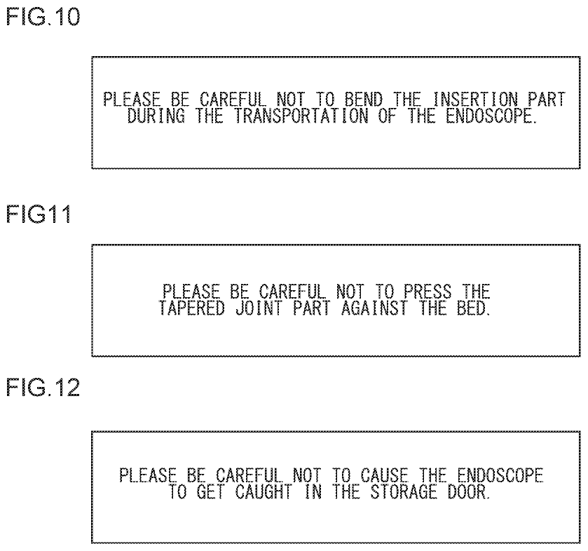

FIG. 10 shows an example of cautionary information presented to a nurse or a cleaner. The cautionary information presentation unit 120 displays the cautionary information regarding “bending the insertion part at a small radius of curvature” on the system screen in a superimposed manner, for example, when the nurse and the cleaner log in to the system. This allows the nurse and the cleaner to reconfirm that the insertion part should not be bent during the transportation of the endoscope, and thus allows for a reduction in the frequency of occurrences of failure caused due to bending the insertion part during transportation.

On Nov. 3, 2018, which is 244 days after Mar. 4, 2018, the cautionary information presentation unit 120 presented the cautionary information regarding “pressing the tapered joint part against the bed” when the doctor checked patient information for the first examination of the day.

FIG. 11 shows an example of cautionary information presented to a doctor. The cautionary information presentation unit 120 displays the cautionary information regarding “pressing the tapered joint part against the bed” on a patient checking screen when the doctor checks the patient information before the first examination on the day. This allows the doctor to reconfirm that the tapered joint part should not be pressed against the bed, thus reducing the frequency of occurrences of failure caused due to pressing the tapered joint part against the bed during the examination.

On Aug. 27, 2018, which is 109 days after May 10, 2018, the cautionary information presentation unit 120 presented cautionary information regarding “getting caught in the storage door” when the nurse first stored an endoscope in the storage on the day.

FIG. 12 shows an example of cautionary information presented to a nurse. The cautionary information presentation unit 120 displays cautionary information regarding “getting caught in the storage door” on the nurse's tablet or the like when the nurse first stores the endoscope in the storage. This allows the nurse to reconfirm that the endoscope should not get caught in the storage door, thus reducing the frequency of occurrences of failure caused due to the endoscope getting caught at the time of storage.

Described above is an explanation on the present disclosure based on the embodiments. These embodiments are intended to be illustrative only, and it will be obvious to those skilled in the art that various modifications to constituting elements and processes could be developed and that such modifications are also within the scope of the present disclosure.

What is claimed is:

1. A medical support apparatus comprising: a processor comprising hardware; and a memory that stores information regarding a period until failure of an endoscope in a plurality of medical facilities for each cause of failure,
wherein the processor is configured to:
identify a second medical facility similar to a first medical facility;
use information regarding a period until failure of an endoscope in the second medical facility so as to determine designated date information regarding a date on which alerting should be made in the first medical facility according to the cause of the failure; and
transmit the combination of the determined designated date information and information regarding the cause of the failure, to the first medical facility.

2. The medical support apparatus according to claim 1,
wherein the processor is configured to use the number of endoscopes that can be used in medical facilities and the model information of the endoscopes that can be used so as to calculate the degree of similarity between the first medical facility and other medical facilities and identify the second medical facility with a high calculated degree of similarity.

3. The medical support apparatus according to claim 1,
wherein the processor is configured to identify a plurality of second medical facilities similar to the first medical facility.

4. The medical support apparatus according to claim 1,
wherein the period until the failure of an endoscope is an interval from the latest alerting date to the latest failure date.

5. The medical support apparatus according to claim 1,
wherein the period until the failure of an endoscope is an interval between two failure dates due to the same cause.

6. An information processor provided in a first medical facility, comprising: a processor comprising hardware,
wherein the processor is configured to:
acquire the combination of designated date information regarding a date on which alerting should be made in the first medical facility and information regarding a cause of the failure, the designated date information which is determined by a medical support apparatus by using information regarding a period until failure of an endoscope in a second medical facility similar to the first medical facility; and
present cautionary information regarding the cause of the failure to a worker on a date specified by the acquired designated date information.

7. A medical support method comprising:
storing information regarding a period until failure of an endoscope in a plurality of medical facilities for each cause of failure;
identifying a second medical facility similar to a first medical facility;
using information regarding a period until failure of an endoscope in the second medical facility so as to determine designated date information regarding a date on which alerting should be made in the first medical facility according to the cause of the failure; and
transmitting the combination of the determined designated date information and information regarding the cause of the failure to the first medical facility.

8. The medical support method according to claim 7,
wherein the identifying of the second medical facility is to use the number of endoscopes that can be used in medical facilities and the model information of the endoscopes that can be used so as to calculate the degree of similarity between the first medical facility and other medical facilities and identify the second medical facility with a high calculated degree of similarity.

9. The medical support method according to claim 7,
wherein the identifying of the second medical facility is to identify a plurality of second medical facilities similar to the first medical facility.

10. The medical support method according to claim 7,
wherein the period until the failure of an endoscope is an interval from the latest alerting date to the latest failure date.

11. The medical support method according to claim 7,
wherein the period until the failure of an endoscope is an interval between two failure dates due to the same cause.
